(19) United States
(12) Patent Application Publication
(10) Pub. No.: US 2005/0130987 A1
(43) Pub. Date: Jun. 16, 2005

(54) Methods of treating vasomotor symptoms

(75) Inventors: Darlene Coleman Deecher, Quakertown, PA (US); Chad Edward Beyer, Newtown, PA (US); Liza Leventhal, Wayne, PA (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 10/962,897

(22) Filed: Oct. 12, 2004

Related U.S. Application Data
(60) Provisional application No. 60/510,897, filed on Oct. 14, 2003.

(30) Foreign Application Priority Data
Oct. 15, 2003 (WO) PCT/US03/32759

(51) Int. Cl. A61K 31/496; A61K 31/4178

(52) U.S. Cl. 514/253.05; 514/397

(57) ABSTRACT

The present invention relates to selective adrenergica_{2B} antagonists alone, selective adrenergicα_{2B} antagonists in combination with norepinephrine reuptake inhibitors (NRI) (as a single compound or as a combination of two or more compounds), or selective adrenergicα_{2B} antagonists in combination with dual norepinephrine reuptake inhibitors/serotonin reuptake inhibitors (NRI/SRI) (as a single compound or as a combination of two or more compounds) and methods of their use in the treatment of vasomotor symptoms.

### CROSS REFERENCE TO RELATED APPLICATIONS

This application claims benefit of:
(a) U.S. application No. 60/510,897 filed Oct. 14, 2003; and also claims priority to:
(b) U.S. application Ser. No. 10/685,812 filed Oct. 14, 2003; and
(c) International Application No. PCT/US03/32759 filed Oct. 15, 2003 in English,
   the entire disclosures of each of the above applications are herein incorporated by reference.

### FIELD OF THE INVENTION

The present invention relates to the use of compounds and composition of compounds that modulate norepinephrine levels for the treatment of, inter alia, vasomotor symptoms (VMS). In particular, the present invention relates to the use of compounds and compositions of compounds having adrenergicα_{2B} antagonist activity for the modulation of the norepinephrine system.

### BACKGROUND OF THE INVENTION

Vasomotor symptoms (VMS), referred to as hot flushes and night sweats, are the most common symptoms associated with menopause, occurring in 60% to 80% of all women following natural or surgically-induced menopause. VMS are likely to be an adaptive response of the central nervous system (CNS) to declining sex steroids. To date, the most effective therapies for VMS are hormone-based treatments, including estrogens and/or some progestins. Hormonal treatments are very effective at alleviating VMS, but they are not appropriate for all women. It is well recognized that VMS are caused by fluctuations of sex steroid levels and can be disruptive and disabling in both males and females. A hot flush can last up to thirty minutes and vary in its frequency from several times a week to multiple occurrences per day. The patient experiences a hot flush as a sudden feeling of heat that spreads quickly from the face to the chest and back and then over the rest of the body. It is usually accompanied by outbreaks of profuse sweating. It may sometimes occur several times an hour, and it often occurs at night. Hot flushes and outbreaks of sweats occurring during the night can cause sleep deprivation. Psychological and emotional symptoms observed, such as nervousness, fatigue, irritability, insomnia, depression, memory loss, headache, anxiety, nervousness or inability to concentrate are considered to be caused by the sleep deprivation following hot flush and night sweats (Kramer et al., In: Murphy et al., 3^{rd} Int'l Symposium on Recent Advances in Urological Cancer Diagnosis and Treatment-Proceedings, Paris, France: SCI: 3-7 (1992)).

Hot flushes may be even more severe in women treated for breast cancer for several reasons: (1) many survivors of breast cancer are given tamoxifen, the most prevalent side effect of which is hot flush; (2) many women treated for breast cancer undergo premature menopause from chemotherapy; (3) women with a history of breast cancer have generally been denied estrogen therapy because of concerns about potential recurrence of breast cancer (Loprinzi, et al., Lancet, 2000, 356(9247): 2059-2063).

Men also experience hot flushes following steroid hormone (androgen) withdrawal. This is true in cases of age-associated androgen decline (Katovich, et al., Proceedings of the Society for Experimental Biology & Medicine, 1990, 193(2): 129-35) as well as in extreme cases of hormone deprivation associated with treatments for prostate cancer (Berendsen, et al., European Journal of Pharmacology, 2001, 419(1): 47-54. As many as one-third of these patients will experience persistent and frequent symptoms severe enough to cause significant discomfort and inconvenience.

The precise mechanism of the VMS is unknown but generally is thought to represent disturbances to normal homeostatic mechanisms controlling thermoregulation and vasomotor activity (Kronenberg et al., “Thermoregulatory Physiology of Menopausal Hot Flashes: A Review,” Can. J. Physiol. Pharmacol., 1987, 65:1312-1324).

The fact that estrogen treatment (e.g. estrogen replacement therapy) relieves the symptoms establishes the link between these symptoms and an estrogen deficiency. For example, the menopausal stage of life is associated with a wide range of other acute symptoms, as described above, and these symptoms are generally estrogen responsive.

It has been suggested that estrogens may stimulate the activity of both the norepinephrine (NE) and/or serotonin (5-HT) systems (J. Pharmacology & Experimental Therapeutics, 1986, 236(3) 646-652). It is hypothesized that estrogens modulate NE and 5-HT levels providing homeostasis in the thermoregulatory center of the hypothalamus. The descending pathways from the hypothalamus via brainstem/spinal cord and the adrenals to the skin are involved in maintaining normal skin temperature. The action of NE and 5-HT reuptake inhibitors is known to impinge on both the CNS and peripheral nervous system (PNS). The pathophysiology of VMS is mediated by both central and peripheral mechanisms and, therefore, the interplay between the CNS and PNS may account for the efficacy of dual acting SRI/NRIs in the treatment of thermoregulatory dysfunction. In fact, the physiological aspects and the CNS/PNS involvement in VMS may account for the lower doses proposed to treat VMS (Loprinzi, et al., Lancet, 2000, 356:2059-2063; Stearns et al., JAMA, 2003, 289:2827-2834) compared to doses used to treat the behavioral aspects of depression. The interplay of the CNS/PNS in the pathophysiology of VMS and the presented data within this document were used to support the claims that the norepinephrine system could be targeted to treat VMS.

Although patients with VMS are most commonly treated by hormone therapy (orally, transdermally, or via an implant), some patients cannot tolerate estrogen treatment (Berendsen, Maturitas, 2000, 36(3): 155-164, Fink et al., Nature, 1996, 383(6598): 306). In addition, hormone replacement therapy is usually not recommended for women or men with or at risk for hormonally sensitive cancers (e.g. breast or prostate cancer). Thus, non-hormonal therapies (e.g. fluoxetine, paroxetine [SRIs] and clonidine) are being evaluated clinically. WO9944601 discloses a method for decreasing hot flushes in a human female by administering fluoxetine. Other options have been studied for the treatment of hot flushes, including steroids, alpha-adrenergic agonists, and beta-blockers, with varying degree of success (Waldinger et al., Maturitas, 2000, 36(3): 165-168).

It has been reported that α₂-adrenergic receptors play a role in thermoregulatory dysfunctions (Freedman et al., Fertility & Sterility, 2000, 74(1): 20-3). These receptors are located both pre- and post-synaptically and mediate an inhibitory role in the central and peripheral nervous system. There are four distinct subtypes of the adrenergic_{α2} receptors, i.e., are α_{2A}, α_{2B}, α_{2C} and α_{2D} (Mackinnon et al., TIPS, 1994, 15: 119; French, Pharmacol. Ther., 1995, 68: 175). It has been reported that a non-select α₂-adrenoceptor antagonist, yohimbine, induces a flush and an α₂-adrenergic receptor agonist, clonidine, alleviates the yohimbine effect (Katovich, et al., Proceedings of the Society for Experimental Biology & Medicine, 1990, 193(2): 129-35, Freedman et al., Fertility & Sterility, 2000, 74(1): 20-3). Clonidine has been used to treat hot flush. However, using such treatment is associated with a number of undesired side effects caused by high doses necessary to abate hot flush described herein and known in the related arts.

Given the complex multifaceted nature of thermoregulation and the interplay between the CNS and PNS in maintaining thermoregulatory homeostasis, multiple therapies and approaches can be developed to target vasomotor symptoms. The present invention focuses on methods directed to these and other important uses.

### SUMMARY OF THE INVENTION

The present invention provides methods for treating a subject afflicted with vasomotor symptoms.

In one aspect, the invention is directed to methods for treating a vasomotor symptom in a subject in need thereof, comprising the step of:
administering to said subject a composition comprising:
an effective amount of an active ingredient consisting essentially of at least one selective adrenergic_{α2B} receptor antagonist or a pharmaceutically acceptable salt thereof.

In another aspect, the invention is directed to methods for treating a vasomotor symptom in a subject in need thereof, comprising the step of:
administering to said subject a composition comprising:
an effective amount of at least one selective adrenergic_{α2B} receptor antagonist or a pharmaceutically acceptable salt thereof; and
an effective amount of at least one norepinephrine reuptake inhibitor (NRI) or a pharmaceutically acceptable salt thereof.

In yet another aspect, the invention is directed to methods for treating a vasomotor symptom in a subject in need thereof, comprising the step of:
administering to said subject a composition comprising:
an effective amount of at least one selective adrenergic_{α2B} receptor antagonist or a pharmaceutically acceptable salt thereof; and
an effective amount of at least one dual norepinephrine reuptake inhibitor/serotonin reuptake inhibitor (NRI/SRI) or a pharmaceutically acceptable salt thereof.

In yet a further aspect, the invention is directed to pharmaceutical compositions, comprising:
an active ingredient consisting essentially of at least one selective adrenergic_{α2B} receptor antagonist or a pharmaceutically acceptable salt thereof; and
at least one pharmaceutically acceptable carrier.

In another aspect, the invention is directed to pharmaceutical compositions, comprising:
at least one selective adrenergic_{α2B} receptor antagonist or a pharmaceutically acceptable salt thereof;
at least one norepinephrine reuptake inhibitor (NRI) or a pharmaceutically acceptable salt thereof; and
at least one pharmaceutically acceptable carrier.

In yet another aspect, the invention is directed to pharmaceutical compositions, comprising:
at least one selective adrenergic_{α2B} receptor antagonist or a pharmaceutically acceptable salt thereof;
at least one norepinephrine reuptake inhibitor/serotonin reuptake inhibitor (NRI/SRI) or a pharmaceutically acceptable salt thereof; and
at least one pharmaceutically acceptable carrier.

### BRIEF DESCRIPTION OF THE DRAWINGS

The invention can be more fully understood from the following detailed description and the accompanying drawings that form a part of this application.

FIG. 1 is the dose response for imiloxan, an adrenergic_{α2B} receptor antagonist, in morphine-dependent rat model of hot flush (MD model); imiloxan ED₅₀ value=15 mg/kg, sc. * indicates p<0.05 compared to vehicle control (referred to in EXAMPLE 1).

FIG. 2 shows the result of the administration of imiloxan, an adrenergicα_{2B} receptor antagonist, at 2 doses (30 and 60 mg/kg, sc) in telemetry rat model of ovariectomy-induced thermoregulatory dysfunction (referred to in EXAMPLE 2).

FIG. 3 shows the results of the microdialysis studies measuring the effects of imiloxan, an adrenergic_{α2B} receptor antagonist, on extracellular levels of NE and 5-HT in the rat preoptic area of the hypothalamus; imiloxan 10 mg/kg, ip. * indicates p<0.05 compared to vehicle control (referred to in EXAMPLE 3).

### DETAILED DESCRIPTION OF THE INVENTION

Adrenergic_{α2} receptor antagonists are known to induce hot flush. Surprisingly, we have discovered that a selective adrenergic_{α2B} receptor antagonist, when administered alone or when co-administered with a NRI compound, resulted in a dose-dependent abatement of a naloxone-induce flush. Furthermore, we have discovered that the efficacy of an NRI may be potentiated when administered in combination with an adrenergic_{α2B} receptor antagonist. It is believed that the present invention described presents a substantial breakthrough in the field of treatment, alleviation, inhibition, and/or prevention of vasomotor instability and/or symptoms.

The present invention provides methods for treating a subject afflicted with vasomotor symptoms by of recovering the reduced activity of norepinephrine. Norepinephrine activity in the hypothalamus or in the brainstem can be elevated by (i) blocking the activity of the adrenergic_{α2B} receptor with an antagonist and/or (ii) blocking the activity of the NE transporter.

More specifically, the present invention provides methods for treating a subject afflicted with vasomotor symptoms comprising administering selective adrenergicα_{2B} antagonists alone, selective adrenergicα_{2B} antagonists in combination with norepinephrine reuptake inhibitors (NRI) (as a single compound or as a combination of two or more compounds), or selective adrenergicα_{2B} antagonists in combination with dual norepinephrine reuptake inhibitors/serotonin reuptake inhibitors (NRI/SRI) (as a single compound or as a combination of two or more compounds). The invention also includes pharmaceutical compositions comprising, as the active ingredient(s), selective adrenergicα_{2B} antagonists alone, selective adrenergicα_{2B} antagonists in combination with norepinephrine reuptake inhibitors (NRI) (as a single compound or as a combination of two or more compounds), or selective adrenergicα_{2B} antagonists in combination with dual norepinephrine reuptake inhibitors/serotonin reuptake inhibitors (NRI/SRI) (as a single compound or as a combination of two or more compounds).

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and Δ TST mean change in tail skin temperature normalized for 15 minutes baseline TST prior to naloxone-induced flush. “ED₅₀ value” means dose which results in 50% alleviation of the observed condition or effect (50% mean maximum endpoint).
“Tail skin temperature” is abbreviated TST.
“Norepinephrine transporter” is abbreviated NET.
“Human norepinephrine transporter” is abbreviated hNET.
“Serotonin transporter” is abbreviated SERT.
“Human serotonin transporter” is abbreviated hSERT.
“Norepinephrine reuptake inhibitor” is abbreviated NRI.
“Selective norepinephrine reuptake inhibitor” is abbreviated SNRI.
“Serotonin reuptake inhibitor” is abbreviated SRI.
“Selective serotonin reuptake inhibitor” is abbreviated SSRI.
“Norepinephrine” is abbreviated NE.
“Serotonin is abbreviated 5-HT.
“Subcutaneous” is abbreviated sc.
“Intraperitoneal” is abbreviated ip.
“Oral” is abbreviated po.
“High Performance Liquid Chromatography” is abbreviated HPLC.

The terms “vasomotor symptoms,” “vasomotor instability symptoms,” and “vasomotor disturbances” include, but are not limited to, hot flushes (flashes), insomnia, sleep disturbances, mood disorders, irritability, excessive perspiration, night sweats, fatigue, and the like, caused by, inter alia, thermoregulatory dysfunction.

The term “hot flush” is an art-recognized term that refers to an episodic disturbance in body temperature typically consisting of a sudden skin flushing, usually accompanied by perspiration in a subject. The term “hot flush” may be used interchangeably with the terms vasomotor symptoms, vasomotor instability, vasomotor dysfunction, night sweats, vasomotor disturbances, and hot flash.

The term “premenopausal,” as used herein, means before the menopause, the term “perimenopausal,” as used herein, means during the menopause and the term postmenopausal,” as used herein, means after the menopause.

The terms “premature menopause” or “artificial menopause” refer to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

The term “ovariectomy,” as used herein, means removal of an ovary or ovaries and can be effected according to Merchenthaler et al., Maturitas, 1998; 30(3): 307-316.

The term “selective adrenergicα_{2B} receptor antagonist,” as used herein, refers to an adrenergic_{α2} receptor antagonist compound that preferentially binds to the adrenergic_{α2B} receptor relative to the adrenergic_{α2A} receptor or adrenergic_{α2C} receptor, and may be measured by the ratio of the in vitro or in vivo IC₅₀ value for antagonist activity at the adrenergic_{α2B} receptor, divided by the IC₅₀ value for antagonist activity at the adrenergic_{α2A or C}. A selective adrenergic_{α2B} receptor antagonist is any adrenergic_{α2} receptor antagonist for which this ratio is greater than about 1, preferably greater than about 2, more preferably greater than about 5, yet more preferably greater than about 10, still more preferably greater than about 50, and more preferably still greater than about 100.

The term “norepinephrine reuptake inhibitor,” as used herein, refers to a compound that alters the level of norepinephrine (NE) by inhibiting the uptake of NE through neurons of the central and/or peripheral nervous system and/or the peripheral system and that has a selectivity ratio of SERT:NET activity, as measured by the EC₅₀ value or by % specific bound NE uptake for the human transporter, of at least about 1:1. Preferably, the selectivity ratio of SERT:NET does not exceed about 1000:1. Preferably, the selectivity ratio of SERT:NET is greater than about 2:1. More preferably, the selectivity ratio of SERT:NET is greater than about 5:1. Even more preferably, the selectivity ratio of SERT:NET is greater than about 10:1.

The phrase “substantially no serotonin reuptake inhibitor (SRI) activity” refers to a norepinephrine reuptake inhibitor that has a selectivity ratio of SERT:NET activity, as measured by the EC₅₀ value or by % specific bound NE uptake for the human transporter, of greater than about 2:1, preferably, greater than about 5:1, and more preferably, greater than about 10:1.

The terms “norepinephrine reuptake inhibitor/serotonin reuptake inhibitor” and “(NRI/SRI)” as used herein, refers to a single compound having dual activity as a serotonin reuptake inhibitor and as a norepinephrine reuptake inhibitor. As used herein, a compound having a dual activity is a dual acting compound.

As term “treatment,” as used herein, includes preventative (e.g., prophylactic), curative or palliative treatment and “treating” as used herein also includes preventative, curative and palliative treatment.

The term “administering,” as used herein, means either directly administering a compound or composition of the present invention, or administering a prodrug, derivative or analog which will form an equivalent amount of the active compound or substance within the body.

The term “effective amount,” as used herein, refers to an amount effective, at dosages, and for periods of time necessary, to achieve the desired result. In particular, “effective amount” refers to the amount of compound or composition of compounds that would increase norepinephrine levels to compensate in part or total for the lack of steroid availability in subjects subject afflicted with a vasomotor symptom. Varying hormone levels will influence the amount of compound required in the present invention. For example, the pre-menopausal state may require a lower level of compound due to higher hormone levels than the peri-menopausal state.

It will be appreciated that the effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. A therapeutically effective amount is also one in which any toxic or detrimental effects of the components are outweighed by the therapeutically beneficial effects.

Preferably, the compounds of the present invention are administered at a dosage and for a time such that the number of hot flushes is reduced as compared to the number of hot flushes prior to the start of treatment. Such treatment can also be beneficial to reduce the overall severity or intensity distribution of any hot flushes still experienced, as compared to the severity of hot flushes prior to the start of the treatment.

The terms “component”, “drug” or “pharmacologically active agent” or “active agent” or “medicament” are used interchangeably herein to refer to a compound or compounds or composition of matter which, when administered to an organism (human or animal) induces a desired pharmacologic and/or physiologic effect by local and/or systemic action. The component herein may contain norepinephrine reuptake inhibiting activity or combined serotonin reuptake inhibiting activity and the norepinephrine reuptake inhibiting activity. Furthermore, the component herein may contain combined norepinephrine reuptake inhibiting activity and the adrenergic_{α2B} receptor antagonist activity.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

The term “combination therapy” refers to the administration of two or more therapeutic agents or compounds to treat a therapeutic condition or disorder described in the present disclosure, for example hot flush, sweating, thermoregulatory-related condition or disorder, or other. Such administration includes co-administration of these therapeutic agents or compounds in a simultaneous manner, such as in a single compound having NRI/adrenergic_{α2B} receptor antagonist activity or in multiple, separate compounds for each NRI, NRI/SRI or adrenergic_{α2} receptor antagonist activities (including separate compounds administered in a single dosage unit). In addition, such administration also includes use of each type of therapeutic agent in a concurrent manner. In either case, the treatment regimen will provide beneficial effects of the drug combination in treating the conditions or disorders described herein.

The term “subject” or “patient” refers to an animal including the human species that is treatable with the compositions, and/or methods of the present invention. The term “subject” or “subjects” is intended to refer to both the male and female gender unless one gender is specifically indicated. Accordingly, the term “patient” comprises any mammal which may benefit from treatment or prevention of vasomotor disturbances, such as a human, especially if the mammal is female, either in the pre-menopausal, peri-menopausal, or post-menopausal period. Furthermore, the term patient comprises female animals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis. However, the term “patient” is not intended to be limited to a woman.

The term “side effect,” as used herein, refers to a consequence other than the one(s) for which an agent or measure is used, as the adverse effects produced by a drug, especially on a tissue or organ system other then the one sought to be benefited by its administration. In the case, for example, of high doses of NRIs or NRI/SRI compounds alone, the term “side effect” may refer to such conditions as, for example, vomiting, nausea, sweating, and flushes (Janowsky, et al., Journal of Clinical Psychiatry, 1984, 45(10 Pt 2): 3-9).

The term “inhibitor,” as used herein, is intended to comprise any compound, e.g., antibody, small molecule, peptide, oligopeptide, polypeptide, or protein, preferably small molecule or peptide, that exhibits a partial, complete, competitive and/or inhibitory effect on mammalian, preferably the human norepinephrine reuptake or both serotonin reuptake and the norepinephrine reuptake, thus diminishing or blocking, preferably diminishing, some or all of the biological effects of endogenous norepinephrine reuptake or of both serotonin reuptake and the norepinephrine reuptake.

The term “pharmaceutically acceptable salts,” as used herein, refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

Further, the compounds of the present invention may exist in unsolvated as well as in solvated forms with pharmaceutically acceptable solvents such as water, ethanol, and the like. In general, the solvated forms are considered equivalent to the unsolvated forms for the purpose of the present invention.

The term, “prodrug,” as used herein, means a compound which is convertible in vivo by metabolic means (e.g. by hydrolysis) to adrenergic_{α2B} receptor antagonists, NRIs, and NRI/SRIs. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

In one aspect, the invention is directed to methods for treating a vasomotor symptom in a subject in need thereof, comprising the step of:
administering to said subject a composition comprising:
an effective amount of an active ingredient consisting essentially of at least one selective adrenergic_{α2B} receptor antagonist or a pharmaceutically acceptable salt thereof.

In another aspect, the invention is directed to methods for treating a vasomotor symptom in a subject in need thereof, comprising the step of:
administering to said subject a composition comprising:
an effective amount of at least one selective adrenergic_{α2B} receptor antagonist or a pharmaceutically acceptable salt thereof; and
an effective amount of at least one norepinephrine reuptake inhibitor (NRI) or a pharmaceutically acceptable salt thereof.

In certain embodiments, the norepinephrine reuptake inhibitor (NRI) is administered simultaneously with the selective adrenergic_{α2B} receptor antagonist. In certain other embodiments, the norepinephrine reuptake inhibitor (NRI) is administered sequentially with the selective adrenergic_{α2B} receptor antagonist.

In certain embodiments, the selective adrenergic_{α2B} receptor antagonist and the norepinephrine reuptake inhibitor (NRI) are a single compound. In certain embodiments, the selective adrenergic_{α2B} receptor antagonist and the norepinephrine reuptake inhibitor (NRI) are separate compounds.

In certain preferred embodiments, the norepinephrine reuptake inhibitor has substantially no serotonin reuptake inhibitor (SRI) activity.

In yet another aspect, the invention is directed to methods for treating a vasomotor symptom in a subject in need thereof, comprising the step of:
administering to said subject a composition comprising:
an effective amount of at least one selective adrenergic_{α2B} receptor antagonist or a pharmaceutically acceptable salt thereof; and
an effective amount of at least one dual norepinephrine reuptake inhibitor/serotonin reuptake inhibitor (NRI/SRI) or a pharmaceutically acceptable salt thereof.

In certain embodiments, the norepinephrine reuptake inhibitor/serotonin reuptake inhibitor (NRI/SRI) is administered simultaneously with the selective adrenergic_{α2B} receptor antagonist. In certain other embodiments, the norepinephrine reuptake inhibitor/serotonin reuptake inhibitor (NRI/SRI) is administered sequentially with the selective adrenergic_{α2B} receptor antagonist.

In certain embodiments, the selective adrenergic_{α2B} receptor antagonist and the norepinephrine reuptake inhibitor/serotonin reuptake inhibitor (NRI/SRI) are a single compound. In certain embodiments, the selective adrenergic_{α2B} receptor antagonist and the norepinephrine reuptake inhibitor/serotonin reuptake inhibitor (NRI/SRI) are separate compounds.

In yet a further aspect, the invention is directed to pharmaceutical compositions, comprising:
an active ingredient consisting essentially of at least one selective adrenergic_{α2B} receptor antagonist or a pharmaceutically acceptable salt thereof; and
at least one pharmaceutically acceptable carrier.

In another aspect, the invention is directed to pharmaceutical compositions, comprising:
at least one selective adrenergic_{α2B} receptor antagonist or a pharmaceutically acceptable salt thereof;
at least one norepinephrine reuptake inhibitor (NRI) or a pharmaceutically acceptable salt thereof; and
at least one pharmaceutically acceptable carrier.

In yet another aspect, the invention is directed to pharmaceutical compositions, comprising:
at least one selective adrenergic_{α2B} receptor antagonist or a pharmaceutically acceptable salt thereof;
at least one norepinephrine reuptake inhibitor/serotonin reuptake inhibitor (NRI/SRI) or a pharmaceutically acceptable salt thereof; and
at least one pharmaceutically acceptable carrier.

Suitable selective adrenergic_{α2B} receptor antagonist include, but are not limited, to 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof. Imiloxan is a preferred selective adrenergic_{α2B} receptor antagonist.

Suitable norepinephrine reuptake inhibitors (NRI) include, but are not limited to, maprotiline; reboxetine; norpramine, desipramine; nisoxetine; atomoxetine; amoxapine; doxepin; lofepramin; amitryptyline; 1-[1-(3-fluorophenyl)-2-(4-methyl-1-piperazinyl)ethyl]cyclohexanol; 1-[1-(3-chlorophenyl)-2-(4-methyl-1-piperazinyl) ethyl]cyclohexanol; 1-[2-(4-methyl-1-piperazinyl)-1-[3-(trifluoromethyl)-phenyl]ethyl] cyclohexanol; 1-[1-(4-methoxy phenyl)-2-(4-methyl-1-piperazinyl)ethyl]cyclohexanol; 1-[1-(3-chlorophenyl)-2-[4-(3-chlorophenyl)-1-piperazinyl]ethyl]cyclohexanol; 1-[1-(3-methoxyphenyl)-2-[4-phenyl methyl)-1-piperazinyl]ethyl]cyclohexanol; 1-[2-(3-chloro phenyl)1-piperazinyl]-1-[3-methoxyphenyl)ethyl]cyclohexanol; 1-[2-[4-(6-chloro-2-pyrazinyl)-1-piperazinyl]-1-[3-methoxyphenyl)ethyl]cyclohexanol; 1-[2-[4-(phenyl methyl)]-1-piperazinyl]-1-[3-(trifluoromethyl)phenyl]ethyl]cyclohexanol; 1-[1-(3-methoxyphenyl)-2-[4-[3-(trifluoromethyl)-phenyl]-1-piperazinyl]ethyl] cyclohexanol; 1-[1-(4-fluorophenyl)-2-[4-(phenylmethyl)-1-piperazinyl] ethyl] cyclohexanol; 1-[1-(3-methoxyphenyl)-2-[4-[3-(trifluoromethyl)-phenyl]-1-piperazinyl]ethyl]cyclopentanol; 1-[1-(4-fluorophenyl)-2-[4-(phenylmethyl)-1-piperazinyl]ethyl]cyclohexanol; 1-[2-(dimethylamino)-1-(3-trifluoromethyl phenyl)ethyl]cyclohexanol; 1-[1-(3-fluorophenyl)-2-(4-methyl-1-piperazinyl) ethyl]cyclohexanol; 1-[1-(3-chlorophenyl)-2-(dimethylamino)ethyl] cyclohexanol; 1-[2-dimethylamino)-1-(3-trifluoromethylphenyl) ethyl]cyclohexanol; 1-[1-(3-chlorophenyl)-2-piperazin-1-yl-ethyl]-cyclohexanol; or a combination or pharmaceutically acceptable salt thereof

Preferred NRIs include is desipramine and 1-[1-(3-chlorophenyl)-2-(4-methyl-1-piperazinyl)ethyl]cyclohexanol, particularly pure R and S enantiomers of 1-[1-(3-chlorophenyl)-2-(4-methyl-1-piperazinyl)ethyl]cyclohexanol. The dimethyl amine derivatives may be synthesized as described, for example, in U.S. Pat. No. 4,535,186, the disclosure of which is incorporated herein by reference in its entirety. The piperazine derivatives may be synthesized as described, for example, in U.S. Pat. No. 4,826,844, the disclosure of which is incorporated herein by reference in its entirety.

Suitable dual norepinephrine reuptake inhibitor/serotonin reuptake inhibitor (NRI/SRI) compounds are venlafaxine, O-desmethyl-venlafaxine (DVS-233 or ODV), milnacipran, duloxetine, and combinations and pharmaceutically acceptable salts thereof. In the case of dual acting NRI/SRI compounds, the compound may exhibit more SRI, approximately equivalent NRI and SRI activity, or preferably more NRI activity.

Within the present invention, the adrenergic_{α2B} receptor antagonists, NRIs, and NRI/SRls may be prepared in the form of pharmaceutically acceptable salts, solvates, and prodrugs.

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, N.Y., 1962); Wilen, S. H. Tables of Resolving Agents and Optical Resolutions, p. 268 (E. L. Eliel, Ed., University of Notre Dame Press, Notre Dame, Ind. 1972).

The compositions of the present invention contain, as active ingredient(s), selective adrenergicα_{2B} antagonists alone, selective adrenergicα_{2B} antagonists in combination with norepinephrine reuptake inhibitors (NRI) (as a single compound or as a combination of two or more compounds), or selective adrenergicα_{2B} antagonists in combination with dual norepinephrine reuptake inhibitors/serotonin reuptake inhibitors (NRI/SRI) (as a single compound or as a combination of two or more compounds). The compositions of the present invention may also comprise at least one pharmaceutically acceptable carrier, adjuvants, and/or excipients, and the like (collectively referred to herein as “pharmaceutically acceptable carrier”) that are well known in the art, for example as described in the Hand Book of Pharmaceutical Excipients, second edition, American Pharmaceutical Association, 1994 (incorporated herein by reference).

The selective adrenergicα_{2B} antagonist compound may be administered either alone or in combination therapy with the norepinephrine reuptake inhibitors (NRI) or the norepinephrine reuptake inhibitor/serotonin reuptake inhibitor compounds (NRI/SRI) to patients in the daily dosage range of from about 0.1 to about 500 mg per day for a time sufficient to reduce and/or substantially eliminate the number and/or severity of hot flushes. A more preferred range is about 1 to about 300 mg per day, the most preferred daily dosage being about 10 to about 200 mg per day.

The norepinephrine reuptake inhibitors (NRI) may be administered in combination therapy with the selective adrenergicα_{2B} antagonist compound to patients in the daily dosage range of from about 0.1 mg/day to about 500 mg/day for a time sufficient to reduce and/or substantially eliminate the number and/or severity of hot flushes. A more preferred range is about 1 mg/day to about 300 mg/day, the most preferred daily dosage being about 1 mg/day to about 200 mg/day.

The norepinephrine reuptake inhibitor/serotonin reuptake inhibitor compounds (NRI/SRI) in combination therapy with the selective adrenergicα_{2B} antagonist compound may be administered to patients in the daily dosage range of from about 0.10 mg/day to about 200 mg/day for a time sufficient to reduce and/or substantially eliminate the number and/or severity of hot flushes. A more preferred range is about 1 mg/day to about 100 mg/day, the most preferred daily dosage being about 10 mg/day to about 50 mg/day.

The selective adrenergicα_{2B} antagonist compound alone or combination therapies of this invention, such as pharmaceutical compositions comprising selective adrenergicα_{2B} antagonists in combination with norepinephrine reuptake inhibitors (NRI), or selective adrenergicα_{2B} antagonists in combination with dual norepinephrine reuptake inhibitors/serotonin reuptake inhibitors (NRI/SRI), may be in any dosage form, such as those described herein, and can also be administered in various ways, as described herein. In a preferred embodiment, the combination products of the invention are formulated together, in a single dosage form (that is, combined together in one capsule, tablet, powder, or liquid, etc.). When the combination products are not formulated together in a single dosage form, the selective adrenergicα_{2B} antagonists in combination with norepinephrine reuptake inhibitors (NRI), or selective adrenergicα_{2B} antagonists in combination with dual norepinephrine reuptake inhibitors/serotonin reuptake inhibitors (NRI/SRI) may be administered at the same time or simultaneously (that is, together), or in any order. When not administered at the same time or simultaneously, that is, when administered sequentially, preferably the administration of selective adrenergicα_{2B} antagonists in combination with norepinephrine reuptake inhibitors (NRI), or selective adrenergicα_{2B} antagonists in combination with dual norepinephrine reuptake inhibitors/serotonin reuptake inhibitors (NRI/SRI) occurs less than about one hour apart, more preferably less than about 30 minutes apart, even more preferably less than about 15 minutes apart, and still more preferably less than about 5 minutes apart.

The pharmaceutical compositions of the invention are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds and composition of the invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

Generally, the active ingredient(s), (selective adrenergicα_{2B} antagonist, NRI, or NRI/SRI or a pharmaceutically acceptable salt thereof) will be present at a level of from about 0.1%, by weight, to about 90% by weight, based on the total weight of the pharmaceutical composition, and pharmaceutically acceptable carrier, if present, will be present at a level of from about 0.1%, by weight, to about 90% by weight, based on the total weight of the pharmaceutical composition. Preferably, the active ingredient(s), (selective adrenergicα_{2B} antagonist, NRI, or NRI/SRI or a pharmaceutically acceptable salt thereof) will be present at a level of from about 1%, by weight, to about 90% by weight, based on the total weight of the pharmaceutical composition, and pharmaceutically acceptable carrier, if present, will be present at a level of from about 1%, by weight, to about 90% by weight, based on the total weight of the pharmaceutical composition. More preferably, the active ingredient(s), (selective adrenergicα_{2B} antagonist, NRI, or NRI/SRI or a pharmaceutically acceptable salt thereof) will be present at a level of from about 5%, by weight, to about 90% by weight, based on the total weight of the pharmaceutical composition, and pharmaceutically acceptable carrier, if present, will be present at a level of from about 5%, by weight, to about 90% by weight, based on the total weight of the pharmaceutical composition. Even more preferably, the active ingredient(s), (selective adrenergicα_{2B} antagonist, NRI, or NRI/SRI or a pharmaceutically acceptable salt thereof) will be present at a level of from about 10%, by weight, to about 90% by weight, based on the total weight of the pharmaceutical composition, and pharmaceutically acceptable carrier, if present, will be present at a level of from about 10%, by weight, to about 90% by weight, based on the total weight of the pharmaceutical composition. Yet even more preferably, the active ingredient(s), (selective adrenergicα_{2B} antagonist, NRI, or NRI/SRI or a pharmaceutically acceptable salt thereof) will be present at a level of from about 25%, by weight, to about 90% by weight, based on the total weight of the pharmaceutical composition, and pharmaceutically acceptable carrier, if present, will be present at a level of from about 25%, by weight, to about 90% by weight, based on the total weight of the pharmaceutical composition.

The route of administration may be any route, which effectively transports the active ingredient(s) to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment.

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley lnterscience, New York, 1981); Wilen, S. H., et al., Tetrahedron, 33:2725 (1977); Eliel, E. L. Stereochemistry of Carbon Compounds, (McGraw-Hill, N.Y., 1962); Wilen, S. H. Tables of Resolving Agents and Optical Resolutions, p. 268 (E. L. Eliel, Ed., University of Notre Dame Press, Notre Dame, Ind. 1972).

The route of administration may be any route, which effectively transports the active norepinephrine reuptake inhibitor(s) or serotonin reuptake inhibitor(s) and norepinephrine reuptake inhibitor(s) to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of norepinephrine reuptake inhibitor(s) and serotonin reuptake inhibitor(s) may be concurrent or simultaneous.

### EXAMPLES

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### General Methods

Reagents: Imiloxan can be purchased commercially (TocrisCookson Inc, Ellisville, Mo.) Desipramine can be prepared as described in U.S. Pat. No. 3,454,554. The following reagents were purchased commercially: morphine alkaloid pellets (Murty Pharmaceuticals, Lexington, Ky.),ketamine (Phoenix Pharmaceuticals, Belmont, Calif.), and naloxone (Research Biochemicals International, St. Louis, Mo.).

Dosing: All doses were prepared based on mg/kg. Compounds were dissolved in either sterile water, or 2.0% Tween/methylcellulose and injected subcutaneously (sc) or intraperitoneally (ip) and used at the following dosages: imiloxan (1, 10, 15 and 30 mg/kg), desipramine (0.01, 1.0, 10, and 30 mg/kg), Ketamine (Ketaject, Phoenix Pharmaceuticals, Belmont, Calif.) was injected intramuscularly in the hindlimb at a dosage (40 mg/kg) that was determined to be mildly sedative but did not cause a change in tail skin temperature.

Animals: Ovariectomized Sprague-Dawley rats (180-220 g) were obtained from a commercial vendor (Taconic, Germantown, N.Y.) and individually housed under 12 hours light/dark cycle in a room maintained at 25° C. For microdialysis experiments, male Sprague-Dawley rats (275-350 g) were obtained from Charles River (Wilmington, Mass.) and group housed until time of surgery. Animals were provided with standard rat chow and water ad libitum.

Morphine-dependent model: Ovariectomized rats were injected once daily for 8-9 days with vehicle to minimize stress responses and then administered compound(s) on test day. On day 4 of dosing, morphine dependence was induced by sc implantation of two slow-release morphine pellets (75 mg/pellet) in the dorsal scapular region. This model is based upon an established morphine-dependent naloxone-induced flush paradigm that is reversible by estrogen treatment (Katovich et al., Proceedings of the Society for Experimental Biology & Medicine, 1990. 193(2): p. 129-35). Four to six days after implantation, morphine withdrawal was induced with an opioid antagonist (naloxone) that causes a transient increase in TST. In a typical experiment, rats were administered their final dose of test compound 40-60 minutes prior to naloxone injection. Rats were mildly sedated with ketamine and a thermistor connected to a MacLab data acquisition system was taped to the base of the tail. Tail skin temperature was then monitored continuously for 35 minutes to establish a baseline temperature. Naloxone was subsequently administered and TST was measured for an additional 35-60 minutes (total recording time 70-95 minutes).

Telemetry model: This model has been modified from a previously reported protocol describing estrogen regulation of diurnal TST patterns (Berendsen et al., 2001). Over a 24 hour period, intact cycling rats decrease TST during the active (dark) phase and TST remains elevated during the inactive (light) phase. In OVX rats, TST is elevated over the entire 24 hour period, thus the usual decrease in TST during the active (dark) phase is lost, thus, a compound's ability to restore this lowering of TST during the active phase was examined. A temperature and physical activity transmitter (PhysioTel TA1OTA-F40, Data Sciences International) was implanted subcutaneously in the dorsal scapular region and the tip of the temperature probe was tunneled subcutaneously 2.5 cm beyond the base of the tail. After a 7-day recovery period, TST readings were continuously recorded for the remainder of the study. Tail skin temperature readings were collected from each animal every 5 minutes with values obtained over a 10 second sampling period. The day before test day, an average baseline TST value was calculated for each animal by averaging temperature readings recorded during the 12 hours active (dark) phase. In these studies, animals were dosed approximately 1 hour prior to the onset of dark cycle.

In vivo microdialysis: Previous work using these microdialysis procedures has been reported (Beyer et al, 2002). Using 2-3% halothane (Fluothane; Zeneca, Cheshire, UK) anesthesia, animals were secured in a stereotaxic frame with ear and incisor bars (David Kopf, Tujunga, Calif.). A microdialysis guide cannula (CMA/12; CMA Microdialysis, Stockholm, Sweden) was directed toward the preoptic area of the hypothalamus using the following coordinates: −0.4 mm anterior to bregma, −1.0 mm lateral from the midline and −6.9 mm ventral to dura with a flat skull (Paxinos and Watson, 1986). Guide cannulae were fixed to the skull with two stainless-steel screws (Small Parts, Roanoke, Va.) and dental acrylic (Plastics One, Roanoke, Va.). Following surgery, animals were individually housed in acrylic cages (45 cm sq.) for approximately 24 hours and had access to food and water ad libitum.

Microdialysis probes (CMA/12; 20 kD cut-off) were purchased from CMA/Microdialysis and consisted of a 2-mm active membrane (OD 0.5 mm) and 14-mm stainless steel shaft (OD 0.64 mm). Probes were perfused with artificial CSF (aCSF; 125 mM NaCl, 3 mM KCI, 0.75 mM MgSO₄ and 1.2 mM CaCl₂, pH 7.4) according to manufacturer's specifications. On the day of experiments, microdialysis probes were inserted, via the guide cannula, into the hypothalamus and perfused with aCSF at a flow rate of 1 μl/min. A 3-hour stabilization period was allowed following probe insertion after which time dialysate was collected every 20 minutes into plastic tubes (CMA) located in a CMA/142 microfraction collector.

Initially, 6 dialysate samples were taken prior to drug injection to demonstrate a steady baseline. Next, animals received an injection of imiloxan (10 mg/kg, ip) or vehicle and dialysate samples were collected for at least 3 hours post-injection. At the end of the experiment, animals were euthanized and probe placement was verified histologically.

Dialysate (10 μl) containing NE and 5-HT were separated by HPLC (C18 ODS3 column, 150×3.0 mm, Metachem, Torrance, Calif.) and detected using an ANTEC electrochemical detector (ANTEC, Netherlands) set at a potential of 0.65V vs. a Ag/AgCl reference electrode. Mobile phase (0.15 M NaH2PO4, 0.25 mM EDTA, 1.75 mM 1-octane sulphonic acid, 2% isopropanol and 4% methanol, pH=4.8) was delivered by a Jasco PU1580 HPLC pump (Jasco Ltd, Essex, U.K) at a flow rate of 0.5 ml/min. Neurochemical data were compared to an external standard curve and all data was acquired using the Atlas software package (Thermo Labsystems, Beverley, Mass.) for the PC.

Statistical analysis: To analyze changes in TST induced by naloxone in morphine-dependent rats, all data were analyzed using a two factor repeated measure ANOVA for “treatment” and “time”. The model was fit to test whether there were significant differences in the responses between treatment groups. Naloxone administration is designated as time zero and data is then analyzed at 5 minute intervals. The first three readings were averaged and used as baseline TST scores. All data were analyzed as ΔTST (TST for each time point—baseline). Multiple comparisons (LSD p-values) among the treatment groups at each time point were used for the analysis. Efficacy of hot flush abatement was determined by evaluating statistical differences at the peak response time of 15 minutes post-naloxone, when the maximal change in TST is observed. A customized SAS-excel (SAS Institute, Cary, N.C.) application was used applying a four parameter logistic model to determine ED₅₀ values. A logistic dose transformation was performed on ΔTST. Maximum flush (ΔTST at 15 minutes post-naloxone) was used in the analysis and the minimum was locked at zero. The ED₅₀ value is reported as the dose of test compound that abates 50% of the naloxone-induced flush. Statisticians in the Biometrics Department (Wyeth Research, Collegeville, Pa.) developed a customized JMP application.

Evaluation of a compound's ability to restore normal lowering of TST in the telemetry model was analyzed using hourly TST values calculated for each animal by averaging the 12 temperature readings obtained every 5 minutes over that recording time. To analyze ΔTST in the telemetry model, a two factors repeated measure ANOVA was performed. The model used for analysis was ΔTST=GRP (group)+HR (hours)+GRP*HR+BASELINE. Thus, the reported least squares means are the expected mean values as if both groups had the same baseline value. Post-hoc tests of hourly GRP*HR samples are t-tests of the difference between groups for each hour. To be conservative, a result was not considered significant unless the p-value was <0.025. All analyses were performed using SAS PROC MIXED (SAS, Carey, N.C.).

The neurochemical effects of imiloxan were analyzed by a two-way analysis of variance (ANOVA) with repeated measures (time). The fmol concentrations of NE and 5-HT during the baseline samples were averaged and this value was denoted as 100%. Subsequent sample values were expressed as a percentage of this preinjection baseline value (% of baseline). Post-hoc analyses were made using the Bonferroni/Dunns adjustment for multiple comparisons. All statistical calculations were performed using the Statview software application (Abacus Concepts Inc., Berkeley, Calif.) for the PC.

### Example 1

### Effect of a Select Adrenergic_{α2B} Receptor Antagonist in Alleviating Vasomotor Instability Using a Naloxone-induced Flush Model in Rats

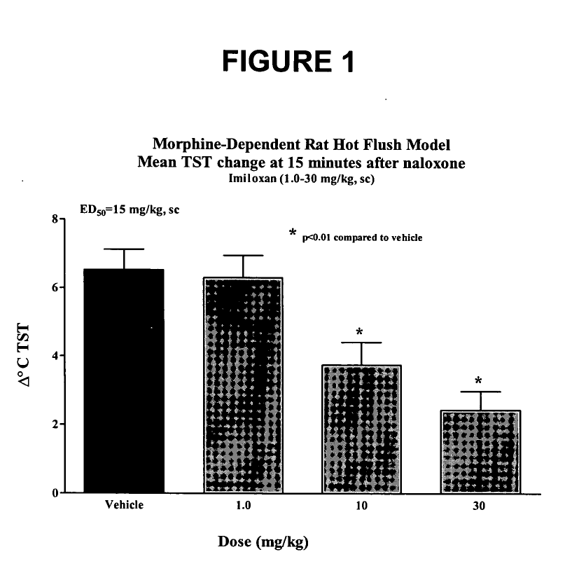

Method used as described in the general method section under morphine-dependent rat model with the following exceptions: Rats were injected subcutaneously with vehicle (2%Tween/0.5% methylcellulose) or imiloxan (Tocris) dissolved in 2%Tween/0.5% methylcellulose and administered subcutaneously at 1.0, 10 and 30 mg/kg 1 hour prior to naloxone. The results are shown in FIG. 1. At maximal flush (15 minutes post-naloxone; Δ° C. TST, Mean+SEM) imiloxan dose-dependently abated the naloxone-induced flush with an estimated ED₅₀ value of 15 mg/kg, sc.

### Example 2

### Effect of a Select Adrenergic_{α2B} Receptor Antagonist in Alleviating Vasomotor Instability Using a Naloxone-induced Flush Model in Rats

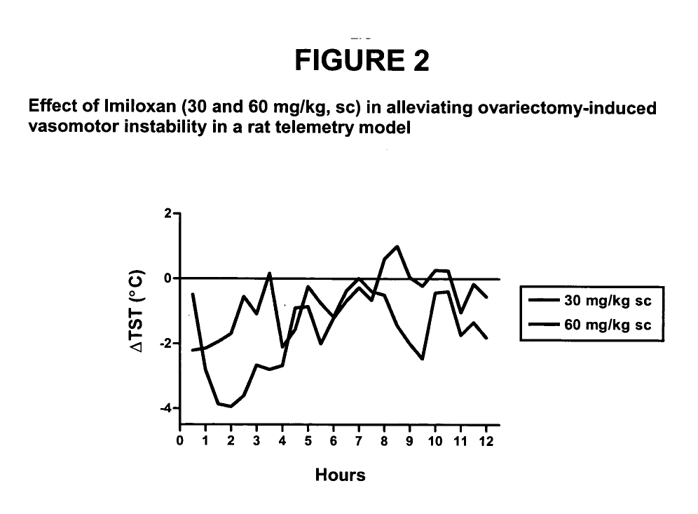

Method used as described in the general method section under telemetry rat model with the following exceptions: Rats were injected subcutaneously with vehicle (2%Tween/0.5% methylcellulose) or 30 or 60 mg/kg, sc imiloxan (Tocris) dissolved in 2%Tween/0.5% methylcellulose. The effect of imiloxan was measured by evaluating the following parameters in this model: onset of action, duration of effect on TST, maximal change in TST and mean change in TST over the duration of the imiloxan's effect. The results are shown in FIG. 2.

Imiloxan (adrenergicα_{2B} receptor antagonist) restored normal TST in an OVX-induced thermoregulatory dysfunction telemetry model (telemetry model) 30 mg/kg, sc * indicates p<0.05 compared to vehicle control.

### Example 3

### Effect of Compounds with Adrenergicα_{2B} Receptor Antagonist Activity and NRI Activity

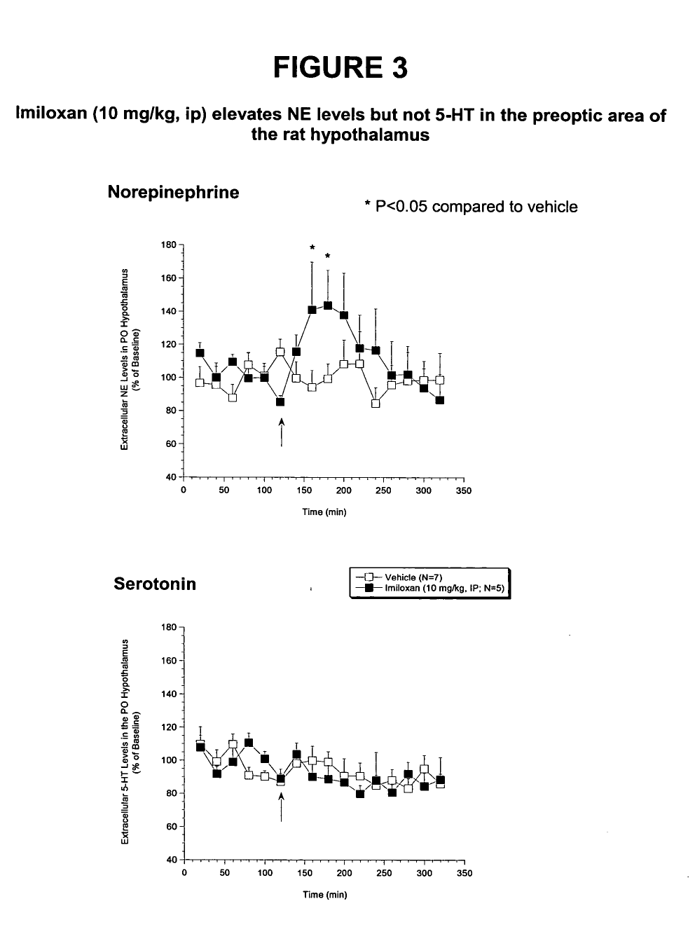

Method used as described in the general method section under morphine-dependent rat model with the following exceptions: Rats were injected subcutaneously with vehicle (2%Tween/0.5% methylcellulose) or imiloxan, 15 mg/kg, sc, or desipramine, 1 mg/kg, sc, dissolved in 2%Tween/0.5% methylcellulose 40 minutes prior to a naloxone-induced flush (maximal flush (15 minutes post-naloxone; Δ° C., Mean+SEM) the combination of imiloxan and desipramine effectively abated the induced flush. The results are shown in FIG. 3.

An additive effect of an adrenergicα_{2B} receptor antagonist (imiloxan) in combination with an NRI (desipramine) on a naloxone-induced flush in the MD model was observed.

### Example 4

### Neurochemical Effect of a Select Adrenergic_{α2B} Receptor Antagonist on Levels of NE and 5-HT in the Preoptic Area of the Rat Hypothalamus

Method used as described in the general method section under in vivo microdialysis: Rats were injected intraperitoneally with vehicle (water) or imiloxan, 10 mg/kg, and NE and 5-HT levels were monitored via microdialysis and HPLC techniques. Acute imiloxan administration significantly elevated concentrations of NE in the preoptic area of the rat hypothalamus 60 minutes post-injection. Conversely, imiloxan treatment did not affect 5-HT levels in the same rats.

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method for treating a vasomotor symptom in a subject in need thereof, comprising the step of:
administering to said subject a composition comprising:
an effective amount of an active ingredient consisting essentially of at least one selective adrenergic_{α2B} receptor antagonist or a pharmaceutically acceptable salt thereof.

2. A method according to claim 1,
wherein said selective adrenergic_{α2B} receptor antagonist is 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof.

3. A method according to claim 2,
wherein said selective adrenergic_{α2B} receptor antagonist is imiloxan or a pharmaceutically acceptable salt thereof.

4. A method for treating a vasomotor symptom in a subject in need thereof, comprising the step of:
administering to said subject a composition comprising:
an effective amount of at least one selective adrenergic_{α2B} receptor antagonist or a pharmaceutically acceptable salt thereof; and
an effective amount of at least one norepinephrine reuptake inhibitor (NRI) or a pharmaceutically acceptable salt thereof.

5. A method according to claim 4,
wherein said norepinephrine reuptake inhibitor (NRI) is administered simultaneously with said selective adrenergic_{α2B} receptor antagonist.

6. A method according to claim 4,
wherein said norepinephrine reuptake inhibitor (NRI) is administered sequentially with said selective adrenergicα_{2B} receptor antagonist.

7. A method according to claim 4,
wherein said selective adrenergicα_{2B} receptor antagonist and said norepinephrine reuptake inhibitor are a single compound.

8. The method of claim 4,
wherein said norepinephrine reuptake inhibitor has substantially no serotonin reuptake inhibitor (SRI) activity.

9. A method according to claim 4,
wherein said selective adrenergicα_{2B} receptor antagonist is 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof.

10. A method according to claim 9,
wherein said selective adrenergic_{α2B} receptor antagonist is imiloxan or a pharmaceutically acceptable salt thereof.

11. A method according to claim 4,
wherein said norepinephrine reuptake inhibitor (NRI) is maprotiline; reboxetine; norpramine, desipramine; nisoxetine; atomoxetine; amoxapine; doxepin; lofepramin; amitryptyline; 1-[1-(3-fluorophenyl)-2-(4-methyl-1-piperazinyl)ethyl]cyclohexanol; 1-[1-(3-chlorophenyl)-2-(4-methyl- 1-piperazinyl) ethyl]cyclohexanol; 1-[2-(4-methyl-1-piperazinyl)-1-[3-(trifluoromethyl)-phenyl]ethyl] cyclohexanol; 1-[1-(4-methoxy phenyl)-2-[4-methyl-1-piperazinyl)ethyl]cyclohexanol; 1-[1-(3-chlorophenyl)-2-[4-(3-chlorophenyl)-1-piperazinyl]ethyl]cyclohexanol; 1-[1-(3-methoxyphenyl)-2-[4-phenyl methyl)-1-piperazinyl]ethyl]cyclohexanol; 1-[2-(3-chloro phenyl)1-piperazinyl]-1-[3-methoxyphenyl)ethyl]cyclohexanol; 1-[2-[4-(6-chloro-2-pyrazinyl)-1-piperazinyl]-1-[3-methoxyphenyl)ethyl]cyclohexanol; 1-[2-[4-(phenyl methyl)]-1-piperazinyl]-1-[3-(trifluoromethyl)phenyl]ethyl]cyclohexanol; 1-[1-(3-methoxyphenyl)-2-[4-[3-(trifluoromethyl)-phenyl]-1-piperazinyl]ethyl] cyclohexanol; 1-[1-(4-fluorophenyl)-2-[4-(phenylmethyl)-1-piperazinyl] ethyl] cyclohexanol; 1-[1-(3-methoxyphenyl)-2-[4-[3-(trifluoromethyl)-phenyl]-1-piperazinyl]ethyl]cyclopentanol; 1-[1-(4-fluorophenyl)-2-[4-(phenylmethyl)-1-piperazinyl]ethyl]cyclohexanol; 1-[2-(dimethylamino)-1-(3-trifluoromethyl phenyl)ethyl]cyclohexanol; 1-[1-(3-fluorophenyl)-2-(4-methyl-1-piperazinyl) ethyl]cyclohexanol; 1-[1-(3-chlorophenyl)-2-(dimethylamino)ethyl] cyclohexanol; 1-[2-dimethylamino)-1-(3-trifluoromethylphenyl) ethyl]cyclohexanol; 1-[1-(3-chlorophenyl)-2-piperazin-1-yl-ethyl]-cyclohexanol;
or a combination or pharmaceutically acceptable salt thereof.

12. The method according to claim 11,
wherein said norepinephrine reuptake inhibitor is desipramine.

13. The method according to claim 11,
wherein said norepinephrine reuptake inhibitor is 1-[1-(3-chlorophenyl)-2-(4-methyl-1-piperazinyl)ethyl]cyclohexanol or pharmaceutically acceptable salt thereof.

14. A method according to claim 13,
wherein said norepinephrine reuptake inhibitor is a pure enantiomer of 1-[1-(3-chlorophenyl)-2-(4-methyl-1-piperazinyl)ethyl]cyclohexanol.

15. A method for treating a vasomotor symptom in a subject in need thereof, comprising the step of:
administering to said subject a composition comprising:
an effective amount of at least one selective adrenergic_{α2B} receptor antagonist or a pharmaceutically acceptable salt thereof; and
an effective amount of at least one dual norepinephrine reuptake inhibitor/serotonin reuptake inhibitor (NRI/SRI) or a pharmaceutically acceptable salt thereof.

16. A method according to claim 15,
wherein said dual norepinephrine reuptake inhibitor/serotonin reuptake inhibitor (NRI/SRI) is administered simultaneously with said selective adrenergic_{α2B} receptor antagonist.

17. A method according to claim 15,
wherein said dual norepinephrine reuptake inhibitor/serotonin reuptake inhibitor (NRI/SRI) is administered sequentially with said selective adrenergic_{α2B} receptor antagonist.

18. A method according to claim 15,
wherein said selective adrenergic_{α2B} receptor antagonist and said dual norepinephrine reuptake inhibitor/serotonin reuptake inhibitor are a single compound.

19. A method according to claim 15,
wherein said selective adrenergic_{α2B} receptor antagonist is 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof.

20. A method according to claim 19,
wherein said selective adrenergic_{α2B} receptor antagonist is imiloxan or a pharmaceutically acceptable salt thereof.

21. A method according to claim 15,
wherein said dual norepinephrine reuptake inhibitor/serotonin reuptake inhibitor (NRI/SRI) is venlafaxine, O-desmethyl-venlafaxine (DVS-233 or ODV), milnacipran, duloxetine, or a combination or a pharmaceutically salt thereof.

22. A method according to claim 1, 4, or 15,
wherein said vasomotor symptom is hot flush.

23. A method according to claim 22,
wherein said subject is human.

24. A method according to claim 23,
wherein said human is a female.

25. A method according to claim 24,
wherein said female is pre-menopausal.

26. A method according to claim 24,
wherein said female is peri-menopausal.

27. A method according to claim 24,
wherein said female is post-menopausal.

28. A method according to claim 23,
wherein said human is a male.

29. A method according to claim 28,
wherein said male is naturally, chemically or surgically andropausal.

30. A pharmaceutical composition, comprising:
an active ingredient consisting essentially of at least one selective adrenergic_{α2B} receptor antagonist or a pharmaceutically acceptable salt thereof; and
at least one pharmaceutically acceptable carrier.

31. A pharmaceutical composition according to claim 30,
wherein said selective adrenergic_{α2B} receptor antagonist is 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof.

32. A composition according to claim 31,
wherein said selective adrenergic_{α2B} receptor antagonist is imiloxan or a pharmaceutically acceptable salt thereof.

33. A pharmaceutical composition, comprising:
at least one selective adrenergic_{α2B} receptor antagonist or a pharmaceutically acceptable salt thereof;
at least one norepinephrine reuptake inhibitor (NRI) or a pharmaceutically acceptable salt thereof; and
at least one pharmaceutically acceptable carrier.

34. A pharmaceutical composition according to claim 33,
wherein said selective adrenergic_{α2B} receptor antagonist is 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof.

35. A composition according to claim 34,
wherein said selective adrenergic_{α2B} receptor antagonist is imiloxan or a pharmaceutically acceptable salt thereof.

36. A composition according to claim 33,
wherein said norepinephrine reuptake inhibitor (NRI) is maprotiline; reboxetine; norpramine, desipramine; nisoxetine; atomoxetine; amoxapine; doxepin; lofepramin; amitryptyline; 1-[1-(3-fluorophenyl)-2-(4-methyl-1-piperazinyl)ethyl]cyclohexanol; 1-[1-(3-chlorophenyl)-2-(4-methyl-1-piperazinyl) ethyl]cyclohexanol; 1-[2-(4-methyl-1-piperazinyl)-1-[3-(trifluoromethyl)-phenyl]ethyl] cyclohexanol; 1-[1-(4-methoxy phenyl)-2-[4-methyl-1-piperazinyl)ethyl]cyclohexanol; 1-[1-(3-chlorophenyl)-2-[4-(3-chlorophenyl)-1-piperazinyl]ethyl]cyclohexanol; 1-[1-(3-methoxyphenyl)-2-[4-phenyl methyl)-1-piperazinyl]ethyl]cyclohexanol; 1-[2-(3-chloro phenyl)1-piperazinyl]-1-[3-methoxyphenyl)ethyl]cyclohexanol; 1-[2-[4-(6-chloro-2-pyrazinyl)-1-piperazinyl]-1-[3-methoxyphenyl)ethyl]cyclohexanol; 1-[2-[4-(phenyl methyl)]-1-piperazinyl]-1-[3-(trifluoromethyl)phenyl]ethyl]cyclohexanol; 1-[1-(3-methoxyphenyl)-2-[4-[3-(trifluoromethyl)-phenyl]-1-piperazinyl]ethyl] cyclohexanol; 1-[1-(4-fluorophenyl)-2-[4-(phenylmethyl)-1-piperazinyl] ethyl] cyclohexanol; 1-[1-(3-methoxyphenyl)-2-[4-[3-(trifluoromethyl)-phenyl]-1-piperazinyl]ethyl]cyclopentanol; 1-[1-(4-fluorophenyl)-2-[4-(phenylmethyl)-1-piperazinyl]ethyl]cyclohexanol; 1-[2-(dimethylamino)-1-(3-trifluoromethyl phenyl)ethyl]cyclohexanol; 1-[1-(3-fluorophenyl)-2-(4-methyl-1-piperazinyl) ethyl]cyclohexanol; 1-[1-(3-chlorophenyl)-2-(dimethylamino)ethyl] cyclohexanol; 1-[2-dimethylamino)-1-(3-trifluoromethylphenyl) ethyl]cyclohexanol; 1-[1-(3-chlorophenyl)-2-piperazin-1-yl-ethyl]-cyclohexanol; or a combination or pharmaceutically acceptable salt thereof.

37. A composition according to claim 36,
wherein said norepinephrine reuptake inhibitor is desipramine.

38. A composition according to claim 36,
wherein said norepinephrine reuptake inhibitor is 1-[1-(3-chlorophenyl)-2-(4-methyl-1-piperazinyl)ethyl]cyclohexanol or pharmaceutically acceptable salt thereof.

39. A composition according to claim 38,
wherein said norepinephrine reuptake inhibitor is a pure enantiomer of 1-[1-(3-chlorophenyl)-2-(4-methyl-1-piperazinyl)ethyl]cyclohexanol.

40. A pharmaceutical composition, comprising:
at least one selective adrenergic_{α2B} receptor antagonist or a pharmaceutically acceptable salt thereof;
at least one norepinephrine reuptake inhibitor/serotonin reuptake inhibitor (NRI/SRI) or a pharmaceutically acceptable salt thereof; and
at least one pharmaceutically acceptable carrier.

41. A pharmaceutical composition according to claim 40,
wherein said selective adrenergic_{α2B} receptor antagonist is 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof.

42. A composition according to claim 41,
wherein said selective adrenergic_{α2B} receptor antagonist is imiloxan or a pharmaceutically acceptable salt thereof.

43. A composition according to claim 41,
wherein said dual norepinephrine reuptake inhibitor/serotonin reuptake inhibitor (NRI/SRI) is venlafaxine, 0-desmethyl-venlafaxine (DVS-233 or ODV), milnacipran, duloxetine, or a combination or a pharmaceutically salt thereof.

44. A product comprising:
at least one selective adrenergic_{α2B} receptor antagonist or a pharmaceutically acceptable salt thereof; and
at least one norepinephrine reuptake inhibitor (NRI) or norepinephrine reuptake inhibitor/serotonin reuptake inhibitor (NRI/SRI) or a pharmaceutically acceptable salt thereof; as a combined preparation for simultaneous, separate or sequential use in the treatment of a vasomotor symptom in a subject in need thereof.

45. A product according to claim 44,
wherein said selective adrenergic_{α2B} receptor antagonist is 2-(1-ethyl-2-imidazoyl)methyl-1,4-benzodioxan (imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), or a combination or a pharmaceutically salt thereof.

46. A product according to claim 44, wherein said selective adrenergicα_{2B} receptor antagonist is imiloxan or a pharmaceutically acceptable salt thereof.

47. A product according to claim 44,
wherein said norepinephrine reuptake inhibitor (NRI) is maprotiline;
reboxetine; norpramine, desipramine; nisoxetine; atomoxetine; amoxapine;
doxepin; lofepramin; amitryptyline; 1-[1-(3-fluorophenyl)-2-(4-methyl-1-piperazinyl)ethyl]cyclohexanol; 1-[1-(3-chlorophenyl)-2-(4-methyl-1-piperazinyl) ethyl]cyclohexanol; 1-[2-(4-methyl-1-piperazinyl)-1-[3-(trifluoromethyl)-phenyl]ethyl] cyclohexanol; 1-[l-(4-methoxy phenyl)-2-[4-methyl-1-piperazinyl)ethyl]cyclohexanol; 1-[1-(3-chlorophenyl)-2-[4-(3-chlorophenyl)-1-piperazinyl]ethyl]cyclohexanol; 1-[1-(3-methoxypheny phenyl methyl)-1-piperazinyl]ethyl]cyclohexanol; 1-[2-(3-chloro phenyl)1-piperazinyl]-1-[3-methoxyphenyl)ethyl]cyclohexanol; 1-[2-[4-(6-chloro-2-pyrazinyl)-1-piperazinyl]-1-[3-methoxyphenyl)ethyl]cyclohexanol; 1-[2-[4-(phenyl methyl)]-1-piperazinyl]-1-[3-(trifluoromethyl)phenyl]ethyl]cyclohexanol; 1-[1-(3-methoxyphenyl)-2-[4-[3-(trifluoromethyl)-phenyl]-1-piperazinyl]ethyl] cyclohexanol; 1-[1-(4-fluorophenyl)-2-[4-(phenylmethyl)-1-piperazinyl]ethyl] cyclohexanol; 1-[1-(3-methoxyphenyl)-2-[4-[3-(trifluoromethyl)-phenyl]-1-piperazinyl]ethyl]cyclopentanol; 1-[1-(4-fluorophenyl)-2-[4-(phenylmethyl)-1-piperazinyl]ethyl]cyclohexanol; 1-[2-(dimethylamino)-1-(3-trifluoromethyl phenyl)ethyl]cyclohexanol; 1-[1-(3-fluorophenyl)-2-(4-methyl-1-piperazinyl) ethyl]cyclohexanol; 1-[1-(3-chlorophenyl)-2-(dimethylamino)ethyl] cyclohexanol; 1-[2-dimethylamino)-1-(3-trifluoromethylphenyl) ethyl]cyclohexanol; 1-[1-(3-chlorophenyl)-2-piperazin-1-yl-ethyl]-cyclohexanol; or a combination or pharmaceutically acceptable salt thereof.

48. A product according to claim 44,
wherein said norepinephrine reuptake inhibitor is desipramine.

49. A product according to claim 44,
wherein said norepinephrine reuptake inhibitor is 1-[1-(3-chlorophenyl)-2-(4-methyl-1-piperazinyl)ethyl]cyclohexanol or pharmaceutically acceptable salt thereof.

50. A product according to claim 49,
wherein said norepinephrine reuptake inhibitor is a pure enantiomer of 1-[1-(3-chlorophenyl)-2-(4-methyl-1-piperazinyl)ethyl]cyclohexanol.

51. A product according to claim 44,
wherein said dual norepinephrine reuptake inhibitor/serotonin reuptake inhibitor (NRI/SRI) is venlafaxine, O-desmethyl-venlafaxine (DVS-233 or ODV), milnacipran, duloxetine, or a combination or a pharmaceutically salt thereof.

52. A product according to claim 44,
wherein said vasomotor symptom is hot flush.

53. A product according to claim 52,
wherein said subject is human.

54. A product according to claim 53,
wherein said human is a female.

55. A product according to claim 54,
wherein said female is pre-menopausal.

56. A product according to claim 54,
wherein said female is peri-menopausal.

57. A product according to claim 54,
wherein said female is post-menopausal.

58. A product according to claim 53,
wherein said human is a male.

59. A product according to claim 58,
wherein said male is naturally, chemically or surgically andropausal.
